(19) United States
(12) Patent Application Publication
(10) Pub. No.: US 2011/0071401 A1
(43) Pub. Date: Mar. 24, 2011

(54) SYSTEMS AND METHODS FOR MAKING AND USING A STEPPER MOTOR FOR AN INTRAVASCULAR ULTRASOUND IMAGING SYSTEM

(75) Inventors: ROGER N. HASTINGS, MAPLE GROVE, MN (US); KEVIN D. EDMUNDS, HAM LAKE, MN (US); TAT-JIN TEO, SUNNYVALE, CA (US)

(73) Assignee: BOSTON SCIENTIFIC SCIMED, INC., MAPLE GROVE, MN (US)

(21) Appl. No.: 12/566,390

(22) Filed: Sep. 24, 2009

(51) Int. Cl. A61B 8/14

(52) U.S. Cl. 600/467

(57) ABSTRACT

A catheter assembly for an intravascular ultrasound system includes an imaging core disposed in a lumen of a catheter. The imaging core includes a stepper motor that rotates a mirror coupled to a driveshaft. The stepper motor provides step-wise rotation of the driveshaft using a rotatable magnet and at least two magnetic field windings disposed around at least a portion of the magnet. At least one fixed transducer is positioned between the stepper motor and the mirror. The stepper motor permits stepwise rotation of the driveshaft with steps of 3 degrees or less. At least one transducer conductor is electrically coupled to the at least one transducer and in electrical communication with a proximal end of the catheter. At least one motor conductor is electrically coupled to the magnetic field windings and in electrical communication with the proximal end of the catheter.

### TECHNICAL FIELD

The present invention is directed to the area of intravascular ultrasound imaging systems and methods of making and using the systems. The present invention is also directed to intravascular ultrasound systems having an imaging core that includes a stepper motor, as well as methods of making and using the stepper motors, imaging cores, and intravascular ultrasound systems.

### BACKGROUND

Intravascular ultrasound (“IVUS”) imaging systems have proven diagnostic capabilities for a variety of diseases and disorders. For example, IVUS imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical pulses that are delivered to the one or more transducers and transformed to acoustic pulses that are transmitted through patient tissue. Reflected pulses of the transmitted acoustic pulses are absorbed by the one or more transducers and transformed to electric pulses. The transformed electric pulses are delivered to the image processor and converted to an image displayable on the monitor.

### BRIEF SUMMARY

In one embodiment, a catheter assembly for an intravascular ultrasound system includes a catheter, an imaging core, at least one transducer conductor, and at least one motor conductor. The catheter has a longitudinal length, a distal end, and a proximal end. The catheter includes a lumen extending along at least a portion of the catheter. The imaging core has a longitudinal length that is substantially less than the longitudinal length of the catheter. The imaging core is configured and arranged for insertion into the lumen of the catheter and disposition at the distal end of the catheter. The imaging core includes a rotatable driveshaft, a mirror, a stepper motor, and at least one fixed transducer. The rotatable driveshaft has a distal end and a proximal end. The mirror is disposed at the distal end of the driveshaft such that rotation of the driveshaft causes a corresponding rotation of the mirror. The stepper motor is coupled to the proximal end of the driveshaft and configured and arranged to provide step-wise rotation of the driveshaft. The stepper motor includes a rotatable magnet and at least two magnetic field windings disposed around at least a portion of the magnet. The at least one fixed transducer is positioned between the stepper motor and the mirror. The at least one transducer has an aperture defined along a longitudinal axis of the at least one transducer. The aperture is configured and arranged to allow passage of the driveshaft through the at least one transducer to the rotatable mirror. The at least one transducer is configured and arranged for transforming applied electrical signals to acoustic signals, transmitting the acoustic signals, receiving corresponding echo signals, and transforming the received echo signals to electrical signals. The at least one transducer conductor is electrically coupled to the at least one transducer and is in electrical communication with the proximal end of the catheter. The at least one motor conductor is electrically coupled to the magnetic field windings and is in electrical communication with the proximal end of the catheter.

In another embodiment, a catheter assembly for an intravascular ultrasound system includes a catheter, an imaging core, at least one transducer conductor, and at least one motor conductor. The catheter has a longitudinal length, a distal end, and a proximal end. The catheter includes a lumen extending along at least a portion of the catheter. The imaging core has a longitudinal length that is substantially less than the longitudinal length of the catheter. The imaging core is configured and arranged for insertion into the lumen of the catheter and disposition at the distal end of the catheter. The imaging core includes a rotatable driveshaft, at least one transducer, a transformer, at least one imaging core conductor, and a stepper motor. The rotatable driveshaft has a distal end and a proximal end. The at least one transducer is disposed at the distal end of the driveshaft such that rotation of the driveshaft causes a subsequent rotation of the at least one transducer. The at least one transducer is configured and arranged for transforming applied electrical signals to acoustic signals, transmitting the acoustic signals, receiving corresponding echo signals, and transforming the received echo signals to electrical signals. The transformer is disposed at the proximal end of the driveshaft. The at least one imaging core conductor couples the at least one transducer to the transformer. The stepper motor is coupled to the driveshaft between the one or more transducers and the transformer. The stepper motor is configured and arranged to produce step-wise rotation of the driveshaft. The stepper motor includes a rotatable magnet and at least two magnetic field windings disposed around at least a portion of the magnet. The magnet has a longitudinal axis and an aperture defined along at least a portion of the longitudinal axis of the magnet. The at least one transducer conductor is electrically coupled to the transformer and extends to the proximal end of the catheter. The least one motor conductor is electrically coupled to the magnetic field windings and extends to the proximal end of the catheter.

In yet another embodiment, a method for imaging a patient using an intravascular ultrasound imaging system includes inserting a catheter into patient vasculature. The catheter has a longitudinal axis and includes an imaging core disposed in a distal portion of a lumen defined in the catheter. The imaging core is electrically coupled to a control module by at least one conductor. The imaging core has a longitudinal axis and includes at least one transducer, a driveshaft, and a magnet that rotates the driveshaft by application of a current from the control module to at least two magnetic field windings wrapped around at least a portion of the magnet. The transducer emits acoustic signals directed at patient tissue. The rotation of the magnet causes rotation of the driveshaft. The imaging core is positioned in a region to be imaged. An electrical signal is applied to the at least two magnetic field windings to generate rotational acceleration of the magnet for a period of time of acceleration sufficient for the magnet to rotate by a selected amount. An electrical signal is applied to the at least two magnetic field windings to generate rotational deceleration of the magnet for a period of time of deceleration that is equal to the period of time of acceleration. An electrical signal is applied to the at least two magnetic field windings to generate the electrical signal causing the magnet to maintain a fixed position for a period of time. At least one acoustic signal is transmitted from the at least one transducer to patient tissue during the period of time when the magnet is maintained in the fixed position. At least one echo signal is received during the period of time when the magnet is maintained in the fixed position. The application of the electrical signals to the at least two magnetic field windings to generate acceleration, deceleration, and causing the magnet to maintain the fixed position for the period of time, as well as the transmission of the at least one acoustic signal and the reception of the at least one echo signal are repeated until the magnet has rotated at least one 360-degree cycle around the longitudinal axis of the imaging core.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an intravascular ultrasound imaging system, according to the invention;

FIG. 2 is a schematic side view of one embodiment of a catheter of an intravascular ultrasound imaging system, according to the invention;

FIG. 3 is a schematic perspective view of one embodiment of a distal end of the catheter shown in FIG. 2 with an imaging core disposed in a lumen defined in the catheter, according to the invention;

FIG. 4 is a schematic longitudinal cross-sectional view of one embodiment of an imaging core disposed in a distal end of a lumen of a catheter, the imaging core including a motor, one or more stationary transducers, and a rotating mirror, according to the invention;

FIG. 5 is a schematic perspective view of one embodiment of a rotating magnet and associated windings, according to the invention;

FIG. 6 is a schematic perspective view of one embodiment of a three-phase winding geometry configured and arranged for forming a rotating magnetic field around a motor, according to the invention;

FIG. 7 is a schematic side view of one embodiment of a portion of a transducer coupled to a portion of a slotted magnetic field winding, transducer conductors coupled to the transducer extend through one of the slots of the magnetic field winding, according to the invention;

FIG. 8 is a graph showing angular displacement of one embodiment of a one-millimeter diameter stepper motor over time, according to the invention; and

FIG. 9 is a schematic longitudinal cross-sectional view of one embodiment of a distal end of a catheter, the distal end of the catheter including an imaging core with a motor, a transformer, and one or more rotating transducers, according to the invention.

### DETAILED DESCRIPTION

The present invention is directed to the area of intravascular ultrasound imaging systems and methods of making and using the systems. The present invention is also directed to intravascular ultrasound systems having an imaging core that includes a stepper motor, as well as methods of making and using the stepper motors, imaging cores, and intravascular ultrasound systems.

Suitable intravascular ultrasound (“IVUS”) imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,306,561; and 6,945,938; as well as U.S. Patent Application Publication Nos. 20060253028; 20070016054; 20070038111; 20060173350; and 20060100522, all of which are incorporated by reference.

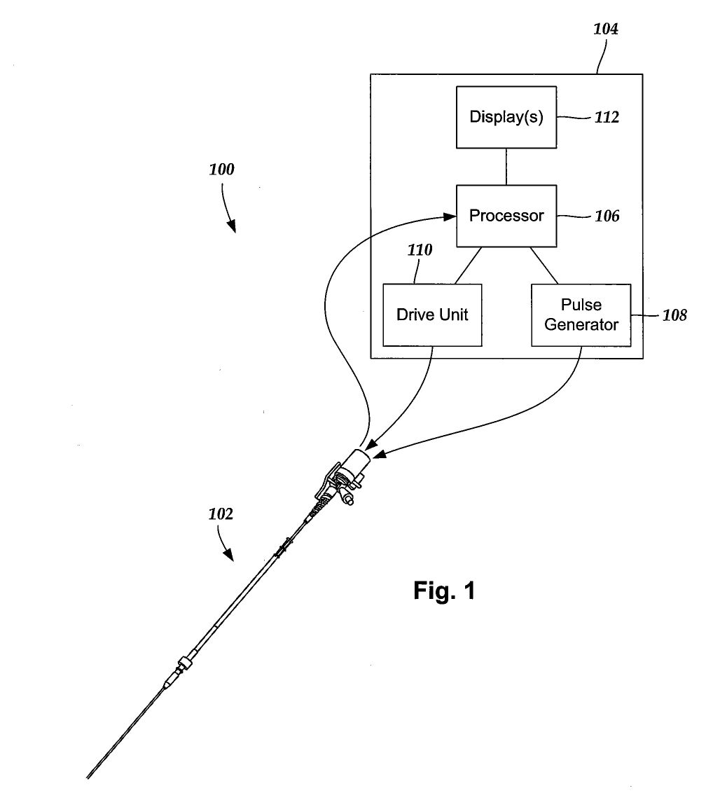

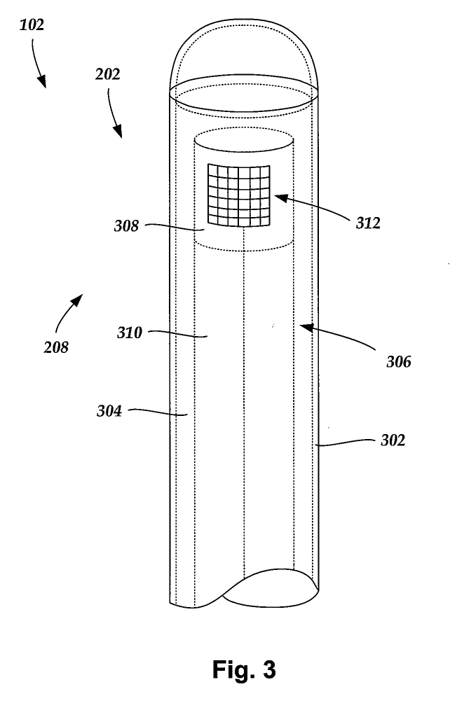

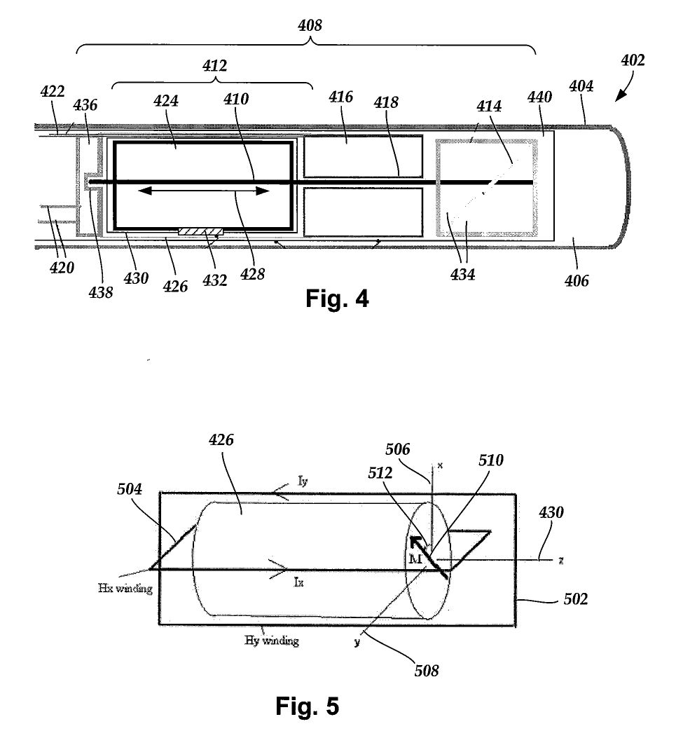

FIG. 1 illustrates schematically one embodiment of an IVUS imaging system 100. The IVUS imaging system 100 includes a catheter 102 that is coupleable to a control module 104. The control module 104 may include, for example, a processor 106, a pulse generator 108, a drive unit 110, and one or more displays 112. In at least some embodiments, the pulse generator 108 forms electric pulses that may be input to one or more transducers (312 in FIG. 3) disposed in the catheter 102. In at least some embodiments, signals from the drive unit 110 may be used to control a motor (see e.g., 416 in FIG. 4) driving an imaging core (306 in FIG. 3) disposed in the catheter 102. In at least some embodiments, electric pulses transmitted from the one or more transducers (312 in FIG. 3) may be input to the processor 106 for processing. In at least some embodiments, the processed electric pulses from the one or more transducers (312 in FIG. 3) may be displayed as one or more images on the one or more displays 112. In at least some embodiments, the processor 106 may also be used to control the functioning of one or more of the other components of the control module 104. For example, the processor 106 may be used to control at least one of the frequency or duration of the electrical pulses transmitted from the pulse generator 108, the rotation rate of the imaging core (306 in FIG. 3) by the motor, the velocity or length of the pullback of the imaging core (306 in FIG. 3) by the motor, or one or more properties of one or more images formed on the one or more displays 112.

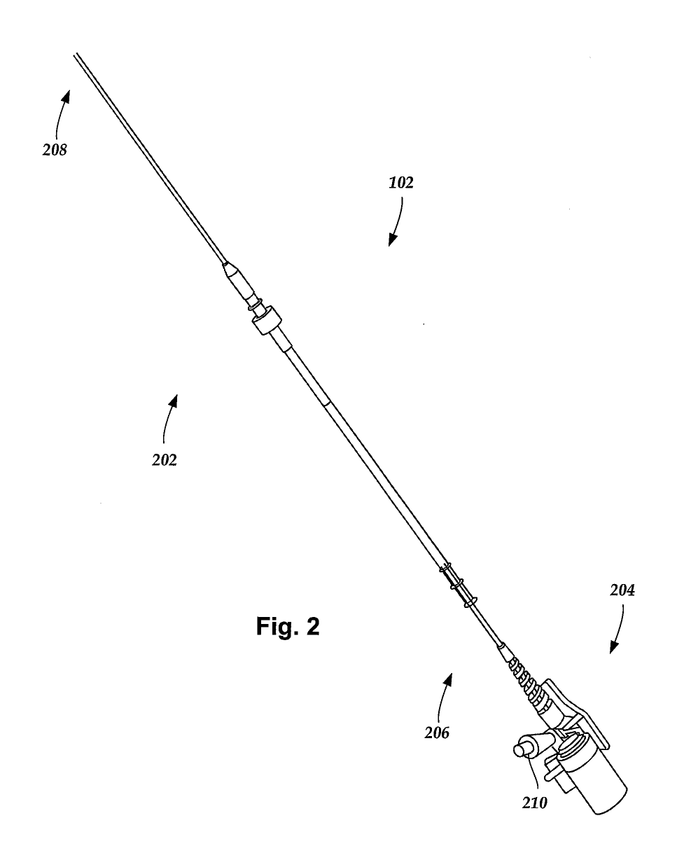

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In at least some embodiments, the catheter 102 defines at least one flush port, such as flush port 210. In at least some embodiments, the flush port 210 is defined in the hub 204. In at least some embodiments, the hub 204 is configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 and a lumen 304. An imaging core 306 is disposed in the lumen 304. The imaging core 306 includes an imaging device 308 coupled to a distal end of a rotatable driveshaft 310.

The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

One or more transducers 312 may be mounted to the imaging device 308 and employed to transmit and receive acoustic pulses. In a preferred embodiment (as shown in FIG. 3), an array of transducers 312 are mounted to the imaging device 308. In other embodiments, a single transducer may be employed. In yet other embodiments, multiple transducers in an irregular-array may be employed. Any number of transducers 312 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used.

The one or more transducers 312 may be formed from one or more known materials capable of transforming applied electrical pulses to pressure distortions on the surface of the one or more transducers 312, and vice versa. Examples of suitable materials include piezoelectric ceramic materials, piezocomposite materials, piezoelectric plastics, barium titanates, lead zirconate titanates, lead metaniobates, polyvinylidenefluorides, and the like.

The pressure distortions on the surface of the one or more transducers 312 form acoustic pulses of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic pulses of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic pulses.

In at least some embodiments, the one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

In at least some embodiments, the imaging core 306 may be rotated about a longitudinal axis of the catheter 102. As the imaging core 306 rotates, the one or more transducers 312 emit acoustic pulses in different radial directions. When an emitted acoustic pulse with sufficient energy encounters one or more medium boundaries, such as one or more tissue boundaries, a portion of the emitted acoustic pulse is reflected back to the emitting transducer as an echo pulse. Each echo pulse that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic pulses transmitted and the echo pulses received. In at least some embodiments, the rotation of the imaging core 306 is driven by the motor (see e.g., 416 in FIG. 4).

As the one or more transducers 312 rotate about the longitudinal axis of the catheter 102 emitting acoustic pulses, a plurality of images are formed that collectively form a radial cross-sectional image of a portion of the region surrounding the one or more transducers 312, such as the walls of a blood vessel of interest and the tissue surrounding the blood vessel. In at least some embodiments, the radial cross-sectional image can be displayed on one or more displays 112.

In at least some embodiments, the imaging core 306 may also move longitudinally along the blood vessel within which the catheter 102 is inserted so that a plurality of cross-sectional images may be formed along a longitudinal length of the blood vessel. In at least some embodiments, during an imaging procedure the one or more transducers 312 may be retracted (i.e., pulled back) along the longitudinal length of the catheter 102. In at least some embodiments, the catheter 102 includes at least one telescoping section that can be retracted during pullback of the one or more transducers 312. In at least some embodiments, the motor (see e.g., 416 in FIG. 4) drives the pullback of the imaging core 306 within the catheter 102. In at least some embodiments, the motor pullback distance of the imaging core is at least 5 cm. In at least some embodiments, the motor pullback distance of the imaging core is at least 10 cm. In at least some embodiments, the motor pullback distance of the imaging core is at least 15 cm. In at least some embodiments, the motor pullback distance of the imaging core is at least 20 cm. In at least some embodiments, the motor pullback distance of the imaging core is at least 25 cm.

The quality of an image produced at different depths from the one or more transducers 312 may be affected by one or more factors including, for example, bandwidth, transducer focus, beam pattern, as well as the frequency of the acoustic pulse. The frequency of the acoustic pulse output from the one or more transducers 312 may also affect the penetration depth of the acoustic pulse output from the one or more transducers 312. In general, as the frequency of an acoustic pulse is lowered, the depth of the penetration of the acoustic pulse within patient tissue increases. In at least some embodiments, the IVUS imaging system 100 operates within a frequency range of 5 MHz to 60 MHz.

In at least some embodiments, one or more conductors 314 electrically couple the transducers 312 to the control module 104 (See FIG. 1). In at least some embodiments, the one or more conductors 314 extend along the catheter 102. In at least some embodiments, a motor may be disposed in the imaging core 308. Examples of IVUS imaging systems with motors disposed in the imaging core 308, for example, U.S. patent application Ser. Nos. 12/415,724; 12/415,768; and 12/415,791, all of which are incorporated by reference.

In at least some embodiments, one or more transducers 312 may be mounted to the distal end 208 of the imaging core 308. The imaging core 308 may be inserted in the lumen of the catheter 102. In at least some embodiments, the catheter 102 (and imaging core 308) may be inserted percutaneously into a patient via an accessible blood vessel, such as the femoral artery, at a site remote from the target imaging location. The catheter 102 may then be advanced through the blood vessels of the patient to the target imaging location, such as a portion of a selected blood vessel.

In at least some embodiments, a rotatable stepper motor (“motor”) is disposed, at least in part, in the imaging core. The motor includes a rotatable magnet driven by a plurality of magnetic field windings. The motor is configured and arranged to rotate such that the motor stops in regular time intervals that are sufficiently long enough for the transducer to transmit an acoustic pulse and receive one or more corresponding echo signals from patient tissue.

The rotatable magnet is disposed in the imaging core. In at least some embodiments, the magnetic field windings (“windings”) are also disposed in the imaging core. In alternate embodiments, the windings are disposed external to the catheter. In at least some embodiments, the windings are disposed external to a patient during an imaging procedure. In at least some embodiments, the imaging core is configured and arranged for insertion into the lumen of the catheter. In at least some embodiments, the imaging core is configured and arranged for extending outward from a distal end of the catheter. In at least some embodiments, the imaging core is configured and arranged for coupling to a guidewire. In at least some embodiments, the imaging core has an outer diameter small enough to allow imaging procedures to be performed from target imaging sites in the brain of a patient, such as one or more of the cerebral arteries.

In at least some embodiments, the imaging core is configured and arranged such that the motor causes a transducer to rotate. In alternate embodiments, the imaging core is configured and arranged such that the motor causes a tilted mirror to rotate while a fixed transducer reflects energy off of a reflective surface of the mirror. An exemplary embodiment of an imaging core with a rotating mirror and fixed transducer is described below, with reference to FIG. 4. An exemplary embodiment of an imaging core with a rotating transducer is described above, with reference to FIG. 3. Additionally, another exemplary embodiment of an imaging core with a rotating transducer is described below, with reference to FIG. 9. It will be understood that the motor may be configured and arranged for rotating the transducer or a mirror or both. Moreover, the rotational attributes of the motor discussed with reference to FIG. 4 apply to the other discussed motors, as well.

FIG. 4 is a schematic longitudinal cross-sectional view of one embodiment of a distal end of a catheter 402. The catheter 402 includes a sheath 404 and a lumen 406. A rotatable imaging core 408 is disposed in the lumen 406 at the distal end of the catheter 402. In at least some embodiments, the imaging core 408 is surrounded by sonolucent fluid. In at least some embodiments, the fluid has an impedance that is within 20 percent of an impedance of patient tissue or fluid at or near a target imaging site within the patient. In at least some embodiments, the fluid has an impedance that is within 15 percent of an impedance of patient tissue or fluid at or near a target imaging site within the patient. In at least some embodiments, the fluid has an impedance that is within 10 percent of an impedance of patient tissue or fluid at or near a target imaging site within the patient. In at least some embodiments, the fluid has an impedance that is within 5 percent of an impedance of patient tissue or fluid at or near a target imaging site within the patient.

The imaging core 408 includes a rotatable driveshaft 410 with a motor 412 and a mirror 414 coupled to the driveshaft 410 and configured and arranged to rotate with the driveshaft 410. The imaging core 408 also includes one or more transducers 416 defining an aperture 418 extending along a longitudinal axis of the one or more transducers 416. In at least some embodiments, the one or more transducers 416 are positioned between the motor 412 and the mirror 414. In at least some embodiments, the one or more transducers 416 are configured and arranged to remain stationary while the driveshaft 410 rotates. In at least some embodiments, the driveshaft 410 extends through the aperture 418 defined in the one or more transducers 416. In at least some embodiments, the aperture 418 is formed from a material, or includes a coating, or both, such as polytetrafluoroethylene coated polyimide tubing, that reduces drag between the rotatable driveshaft 410 and the stationary (relative to the driveshaft 410) aperture 418 of the one or more transducers 416.

One or more motor conductors 420 electrically couple the motor 412 to the control module (104 in FIG. 1). In at least some embodiments, one or more of the motor conductors 420 may extend along at least a portion of a longitudinal length of the catheter 402 as shielded electrical cables, such as a coaxial cable, or a twisted pair cable, or the like. In at least some embodiments, one or more of the motor conductors 420 may be attached to contacts on the distal end of the catheter 402 that, in turn, are connected to control module contacts. One or more transducer conductors 422 electrically couple the one or more transducers 416 to the control module (104 in FIG. 1). In at least some embodiments, one or more of the transducer conductors 422 may extend along at least a portion of the longitudinal length of the catheter 402 as shielded electrical cables, such as a coaxial cable, or a twisted pair cable, or the like. In at least some embodiments, one or more of the transducer conductors 422 may be attached to contacts on the distal end of the catheter 402 that, in turn, are connected to control module contacts.

In at least some embodiments, the outer diameter of the catheter 402 is no greater than 0.042 inches (0.11 cm). In at least some embodiments, the outer diameter of the catheter 402 is no greater than 0.040 inches (0.11 cm). In at least some embodiments, the outer diameter of the catheter 402 is no greater than 0.038 inches (0.10 cm). In at least some embodiments, the outer diameter of the catheter 402 is no greater than 0.036 inches (0.09 cm). In at least some embodiments, the outer diameter of the catheter 402 is no greater than 0.034 inches (0.09 cm). In at least some embodiments, the outer diameter of the catheter 402 is sized to accommodate known intracardiac echocardiography systems.

The motor 412 includes a rotor 424 and a stator 426. In at least some embodiments, the rotor 424 is a permanent magnet with a longitudinal axis 428 (shown in FIG. 4 as a two-headed arrow) that is parallel to a longitudinal axis of the driveshaft 410. The magnet 424 may be formed from any magnetic material suitable for implantation including, for example, neodymium-iron-boron, or the like. One example of a suitable neodymium-iron-boron magnet is available through Hitachi Metals America Ltd, San Jose, Calif.

In at least some embodiments, the outer diameter of the magnet 424 is no greater than 0.025 inches (0.06 cm). In at least some embodiments, the outer diameter of the magnet 424 is no greater than 0.022 inches (0.06 cm). In at least some embodiments, the outer diameter of the magnet 424 is no greater than 0.019 inches (0.05 cm). In at least some embodiments, the longitudinal length of the magnet 424 is no greater than 0.013 inches (0.03 cm). In at least some embodiments, the longitudinal length of the magnet 424 is no greater than 0.012 inches (0.03 cm). In at least some embodiments, the longitudinal length of the magnet 424 is no greater than 0.011 inches (0.03 cm).

In at least some embodiments, the magnet 424 is cylindrical. In at least some embodiments, the magnet 424 has a magnetization M of no less than 1.4 T. In at least some embodiments, the magnet 424 has a magnetization M of no less than 1.5 T. In at least some embodiments, the magnet 424 has a magnetization M of no less than 1.6 T. In at least some embodiments, the magnet 424 has a magnetization vector that is perpendicular to the longitudinal axis 428 of the magnet 424.

In at least some embodiments, the magnet 424 is disposed in a housing 430. In at least some embodiments, the housing 430 is formed, at least in part, from a conductive material (e.g., carbon fiber and the like). In at least some embodiments, the rotation of the magnet 424 produces eddy currents which may increase as the angular velocity of the magnet increases. Once a critical angular velocity is met or exceeded, the eddy currents may cause the magnet to levitate. In a preferred embodiment, the conductive material of the housing 430 has conductivity high enough to levitate the magnet 424 to a position equidistant from opposing sides of the housing 430, yet low enough to not shield the magnet 424 from a magnetic field produced by the stator 426.

In at least some embodiments, a space between the magnet 424 and the housing 430 is filled with a magnetic fluid suspension (“ferrofluid”) (e.g., a suspension of magnetic nano-particles, such as available from the Ferrotec Corp., Santa Clara, Calif.). The ferrofluid is attracted to the magnet 424 and remains positioned at an outer surface of the magnet 424 as the magnet 424 rotates. The fluid shears near the walls of non-rotating surfaces such that the rotating magnet 424 does not physically contact these non-rotating surfaces. In other words, if enough of the surface area of the magnet 424 is accessible by the ferrofluid, the ferrofluid may cause the magnet 424 to float, thereby potentially reducing friction between the magnet 424 and other contacting surfaces which may not rotate with the magnet 424 during operation. In at least some embodiments, the resulting viscous drag torque on the magnet 424 increases in proportion to the rotation frequency of the magnet 424, and may be reduced relative to a non-lubricated design.

The magnet 424 is coupled to the driveshaft 410 and is configured and arranged to rotate the driveshaft 410 during operation. In at least some embodiments, the magnet 424 is rigidly coupled to the driveshaft 410. In at least some embodiments, the magnet 424 is coupled to the driveshaft 410 by an adhesive.

In at least some embodiments, the stator 426 includes at least two perpendicularly-oriented windings (502 and 504 in FIG. 5) which provide a rotating magnetic field to produce torque causing rotation of the magnet 424. The stator 426 is provided with power from the control module (104 in FIG. 1) via the one or more motor conductors 420.

In at least some embodiments, a sensing device 432 is disposed on or near the imaging core 408. In at least some embodiments, the sensing device 432 is coupled to the housing 432. In at least some embodiments, the sensing device 432 is configured and arranged to measure the amplitude of the magnetic field in a particular direction. In at least some embodiments, the sensing device 432 uses at least some of the measured information to sense the angular position of the magnet 424. In at least some embodiments, at least some of the measured information obtained by the sensing device 432 is used to control the current provided to the stator 426 by the one or more motor conductors 420. In at least some embodiments, the sensing device 432 can be used to sense the angular position of the mirror 414.

In at least some embodiments, acoustic signals may be emitted from the one or more transducers 416 towards the rotating mirror 414 and redirected to an angle that is not parallel to the longitudinal axis 428 of the magnet 424. In at least some embodiments, acoustic signals may be redirected to a plurality of angles that are within a 120 degree range with respect to the longitudinal axis 428 of the magnet 424. In at least some embodiments, acoustic signals may be redirected to a plurality of angles that are within a 90 degree range with respect to the longitudinal axis 428 of the magnet 424. In at least some embodiments, acoustic signals may be redirected to a plurality of angles that are within a 120 degree range with respect to the longitudinal axis 428 of the magnet 424 such that the plurality of angles are centered on an angle that is perpendicular to the longitudinal axis 428 of the magnet 424. In at least some embodiments, acoustic signals may be redirected to a single angle that is perpendicular to the longitudinal axis 428 of the magnet 424. In at least some embodiments, acoustic signals may be redirected to a single angle that is not perpendicular to the longitudinal axis 428 of the magnet 424.

In at least some embodiments, the mirror 414 is sandwiched between sonolucent material 434. In at least some embodiments, the sonolucent material is solid or semi-solid. In at least some embodiments, the sonolucent material 434 has an impedance that is within 20 percent of the impedance of the sonolucent fluid surrounding the imaging core 408. In at least some embodiments, the sonolucent material 434 has an impedance that is within 15 percent of the impedance of the sonolucent fluid surrounding the imaging core 408. In at least some embodiments, the sonolucent material 434 has an impedance that is within 10 percent of the impedance of the sonolucent fluid surrounding the imaging core 408. In at least some embodiments, the sonolucent material 434 has an impedance that is within 5 percent of the impedance of the sonolucent fluid surrounding the imaging core 408.

In at least some embodiments, the sonolucent material 434 is disposed over the mirror 414 such that the mirror 414 and sonolucent material 434 form a structure with an even weight distribution around the driveshaft 410. In at least some embodiments, the sonolucent material 434 is disposed over the mirror 414 such that the mirror 414 and sonolucent material 434 form a cylindrically-shaped structure.

In at least some embodiments, the mirror 414 includes a reflective surface that is planar. In at least some embodiments, the mirror 414 includes a reflective surface that is non-planar. In at least some embodiments, the reflective surface of the mirror 414 is concave. It may be an advantage to employ a concaved reflective surface to improve focusing, thereby improving lateral resolution of acoustic pulses emitted from the catheter 402. In at least some embodiments, the reflective surface of the mirror 414 is convex. In at least some embodiments, the shape of the reflective surface of the mirror 414 is adjustable. It may be an advantage to have an adjustable reflective surface to adjust the focus or depth of field for imaging tissues at variable distances from the mirror 414.

In at least some embodiments, the imaging core 108 includes a proximal end cap 436. In at least some embodiments, the proximal end cap 436 provides structure to the proximal portion of the imaging core 108. In at least some embodiments, the proximal end cap 436 is rigid enough to withstand lateral forces (i.e., off-axis forces) typically encountered during normal operation within patient vasculature such that the operation of the motor 412 is not interrupted. In at least some embodiments, a proximal end of the driveshaft 410 contacts the proximal end cap 436. In at least some embodiments, the proximal end cap 436 defines a drag-reducing element 438 for reducing drag caused by the rotating driveshaft 410 contacting the proximal end cap 436. The drag-reducing element 438 can be any suitable device for reducing drag including, for example, one or more bushings, one or more bearings, or the like or combinations thereof.

In at least some embodiments, the catheter 402 includes an inner sheath 440 surrounding the imaging core 408. In at least some embodiments, the inner sheath 440 physically contacts at least one of the motor 412 or the one or more transducers 416, but does not physically contact the rotating mirror 414 during normal operation of the imaging core 408. In at least some embodiments, the inner sheath 440 is rigid. In at least some embodiments, the inner sheath 440 is rigid enough to withstand lateral forces (i.e., off-axis forces) typically encountered during normal operation within patient vasculature such that the mirror 414 does not contact the inner sheath 440. In at least some embodiments, the inner sheath 440 is filled with a sonolucent fluid. In at least some embodiments, the sonolucent fluid has an impedance that is within 20 percent of the impedance of the sonolucent fluid within the lumen 404 of the catheter 402. In at least some embodiments, the sonolucent fluid has an impedance that is within 15 percent of the impedance of the sonolucent fluid within the lumen 404 of the catheter 402. In at least some embodiments, the sonolucent fluid has an impedance that is within 10 percent of the impedance of the sonolucent fluid within the lumen 404 of the catheter 402. In at least some embodiments, the sonolucent fluid has an impedance that is within 5 percent of the impedance of the sonolucent fluid within the lumen 404 of the catheter 402.

In at least some embodiments, the motor 412 provides enough torque to rotate the one or more transducers 416 at a frequency of at least 15 Hz. In at least some embodiments, the motor 412 provides enough torque to rotate the one or more transducers 416 at a frequency of at least 20 Hz. In at least some embodiments, the motor 412 provides enough torque to rotate the one or more transducers 416 at a frequency of at least 25 Hz. In at least some embodiments, the motor 412 provides enough torque to rotate the one or more transducers 416 at a frequency of at least 30 Hz. In at least some embodiments, the motor 412 provides enough torque to rotate the one or more transducers 416 at a frequency of at least 35 Hz. In at least some embodiments, the motor 412 provides enough torque to rotate the one or more transducers 416 at a frequency of at least 40 Hz.

In a preferred embodiment, the torque is about the longitudinal axis 428 of the magnet 424 so that the magnet 424 rotates. In order for the torque of the magnet 424 to be about the longitudinal axis 428 of the magnet 424, the magnetic field generated by the windings (i.e., coils of the stator 426) lies in the plane perpendicular to the longitudinal axis 428 of the magnet 424, with a magnetic field vector rotating about the longitudinal axis 428 of the magnet 424.

As discussed above, the stator 426 provides a rotating magnetic field to produce a torque on the magnet 424. The stator 426 may comprise two perpendicularly-oriented windings that wrap around the magnet 424 as one or more turns to form a rotating magnetic field. FIG. 5 is a schematic perspective view of one embodiment of the rotating magnet 424 and windings, represented as orthogonal rectangular boxes 502 and 504. Although the windings 502 and 504 are shown as two orthogonal rectangles, it will be understood that the each of the windings 502 and 504 may represent multiple turns of wire which may be spread out to minimize an increase in the outer diameter of the catheter (402 in FIG. 4). When the windings 502 and 504 are spread out, a band of current may be generated instead of the lines of current shown in FIG. 5. In at least some embodiments, the windings are formed on a thin film that may be overlaid onto a substrate (e.g., housing 430, or the like).

In preferred embodiments, the stator 426 is formed from rigid or semi-rigid materials using multiple-phase winding geometries. It will be understood that there are many different multiple-phase winding geometries and current configurations that may be employed to form a rotating magnetic field. For example, the stator 426 may include, for example, a two-phase winding, a three-phase winding, a four-phase winding, a five-phase winding, or more multiple-phase winding geometries. It will be understood that a motor may include many other multiple-phase winding geometries. In a two-phase winding geometry, for example, the currents in the two windings are out of phase by 90°. For a three-phase winding, there are three lines of sinusoidal current that are out of phase by zero, 120°, and 240°, with the three current lines also spaced by 120°, resulting in a uniformly rotating magnetic field that can drive a cylindrical rotor magnet magnetized perpendicular to the current lines.

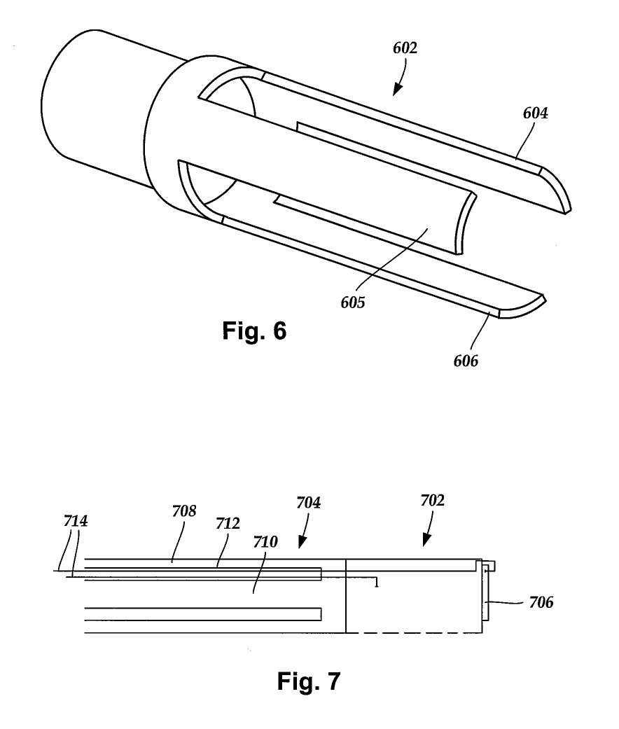

FIG. 6 is a schematic perspective view of one embodiment of a three-phase winding geometry 602 configured and arranged for forming a rotating magnetic field around a magnet (see e.g., 424 in FIG. 4). The three-phase winding 602 includes three arms 604-606 onto which windings can be disposed. In at least some embodiments, multiple windings may utilize a single cylindrical surface of the stator (426 of FIG. 4) with no cross-overs. Such a winding may occupy a minimal volume in an imaging core. Although other geometries may also form a rotating magnetic field, the three-phase geometry 602 may have the advantages of allowing for a more compact motor construction than other geometries.

An exceptional property of a three-phase winding geometry 602 is that only two of the three windings disposed on the arms 604-606 need to be driven, while the third winding is a common return that mathematically is equal to the third phase of current. In at least some embodiments, the arms 604-606 may be supported by a substrate to increase mechanical stability. In at least some embodiments, the arms 604-606 are constructed from a solid metal tube (e.g., a hypotube, or the like), leaving most of the metal in tact, and removing only metal needed to prevent electrical shorting between the lines 604-606. For example, in at least some embodiments, the arms 604-606 are formed from a cylindrical material with a plurality of slits defined along at least a portion of a longitudinal length of each of the arms 604-606, at least some of the slits separating adjacent windings.

FIG. 7 is a schematic side view of one embodiment of a portion of a transducer 702 coupled to a portion of a stator 704. The transducer 702 includes a front face 706 from which acoustic signals may be emitted. The stator 704 includes windings disposed on arms, such as arms 708 and 710 separated from one another by longitudinal slits, such as slit 712 separating arm 708 from arm 710. Transducer conductors 714 electrically couple the transducer 702 to the control module (104 in FIG. 1). In at least some embodiments, the transducer conductors 714 extend along at least a portion of one or more of the slits (such as slit 712) extending along a longitudinal length of the stator 704. It may be an advantage to extend the transducer conductors 714 along one or more of the slits of the stator 704 to potentially reduce the diameter of the imaging core (see e.g., 408 of FIG. 4). In at least some embodiments, at least a portion of the stator 704 extends over at least a portion of the transducer 702. In at least some embodiments, the portion of the stator 704 extending over the portion of the transducer 702 extends such that radial return currents occur far enough distal to the magnet (424 in FIG. 4) to produce only negligible torque on the magnet (424 in FIG. 4).

As discussed above, acoustic pulses are transmitted from the transducer. Echo signals are reflected off patient tissue and sensed by the transducer. When the motor is rotating either the transducer or the mirror during an imaging procedure, the rotating component will have moved some amount in the time between transmitting an acoustic pulse and receiving one or more corresponding echo signals. It would, therefore, be desirable to stop the motor from rotating the transducer or the mirror for the period of time between the transmission of the acoustic pulse and the receival of the corresponding echo signal(s).

Conventional drive shafts and proximal motors may have too much inertia to be able to start and stop fast enough to keep pace with the rate of transmission and reception of energy to and from patient tissue. Additionally, rapid acceleration and deceleration of conventional drive shafts and proximal motors may cause the imaging core to rock when the imaging core starts and stops. As discussed above, in at least some embodiments, transducers (or mirrors) may be configured and arranged to rotate many times per second. Additionally, in at least some embodiments, transducers may emit hundreds, or even thousands or more acoustic pulses during each complete rotation of the transducers (or mirrors).

For example, in at least some embodiments, the magnet 424 is configured and arranged to stepwise rotate at least 200 times during each complete 360-degree cycle of the mirror. In at least some embodiments, the magnet 424 is configured and arranged to stepwise rotate at least 250 times during each complete 360-degree cycle of a transducer or mirror In at least some embodiments, the magnet 424 is configured and arranged to stepwise rotate at least 300 times during each complete 360-degree cycle of a transducer or mirror. In at least some embodiments, the magnet 424 is configured and arranged to stepwise rotate at least 400 times during each complete 360-degree cycle of a transducer or mirror. In at least some embodiments, the magnet 424 is configured and arranged to stepwise rotate at least 500 times during each complete 360-degree cycle of a transducer or mirror. In at least some embodiments, the magnet 424 is configured and arranged to stepwise rotate at least 1000 times during each complete 360-degree cycle of a transducer or mirror.

In at least some embodiments, the magnet 424 is configured and arranged to permit stepwise rotation of the driveshaft 410 every 6 degrees or less. In at least some embodiments, the magnet 424 is configured and arranged to permit stepwise rotation of the driveshaft 410 every 5 degrees or less. In at least some embodiments, the magnet 424 is configured and arranged to permit stepwise rotation of the driveshaft 410 every 4 degrees or less. In at least some embodiments, the magnet 424 is configured and arranged to permit stepwise rotation of the driveshaft 410 every 3 degrees or less. In at least some embodiments, the magnet 424 is configured and arranged to permit stepwise rotation of the driveshaft 410 every 2 degrees or less. In at least some embodiments, the magnet 424 is configured and arranged to permit stepwise rotation of the driveshaft 410 every one degree or less.

By way of example, when a transducer transmits acoustic signals 256 times per revolution and rotates (or reflects off of a rotating mirror that rotates) at 30 Hz, in order for the motor 412 to stop rotation between each acoustic pulse transmission and corresponding echo signal reception the motor 412 stops every 1.4 degrees. If, for example, the motor 412 remains stopped for approximately 30 microseconds, the motor 412 has approximately 100 microseconds between adjacent stops.

In at least some embodiments, the transducer remains stopped for no more than 100 microseconds. In at least some embodiments, the transducer remains stopped for no more than 90 microseconds. In at least some embodiments, the transducer remains stopped for no more than 80 microseconds. In at least some embodiments, the transducer remains stopped for no more than 70 microseconds. In at least some embodiments, the transducer remains stopped for no more than 60 microseconds. In at least some embodiments, the transducer remains stopped for no more than 50 microseconds. In at least some embodiments, the transducer remains stopped for no more than 40 microseconds. In at least some embodiments, the transducer remains stopped for no more than 30 microseconds. In at least some embodiments, the transducer remains stopped for no more than 20 microseconds. In at least some embodiments, the transducer remains stopped for no more than 10 microseconds. In at least some embodiments, the transducer remains stopped for no more than 5 microseconds.

A transducer transmission rate of 256 times per revolution and a rotation frequency of 30 Hz are used above, and also in several examples below, as exemplary values to describe functionality of the motor. It will be understood that the above numbers are each exemplary values and that any motor of the invention can use other values. In at least some embodiments, the one or more transducers 416 transmits more or less than 256 acoustic signals per revolution, and the transducer (or mirror) has a frequency that is higher or lower than 30 Hz. Additionally, it will be understood that the amount of time that the motor 412 remains idle between successive rotations can be adjusted, as desired for a particular application.

As discussed above, the windings generate a magnetic field in a desired direction which causes the magnet to rotate as the magnet aligns with the applied magnetic field. Magnetic torque is the cross product between the magnetic moment of the windings and the applied magnetic field. Thus, the torque goes to zero when the rotor is aligned with the magnetic field. Once aligned, the applied magnetic field provides a restoring force proportional to the angle that the rotor deviates from the direction of the applied magnetic field, thereby maintaining alignment of the rotor.

In order to accommodate the many frequent stops between rotations of the magnet, rapid acceleration of a magnetic field can be used between stops. When the reorientation of the magnetic field is in an increment of only a couple of degrees, however, the new direction may provide a torque that is not sufficiently large enough to produce a rapid acceleration of the rotor. In order to increase torque, the torque may be applied to the magnetic field at right angles to the rotor magnetization vector. When the magnetic field is applied at right angles to the magnetization vector, however, stopping the motor may be difficult.

Assuming that the acceleration torque is substantially greater than frictional drag on the rotor, a motor rotation algorithm may include: applying a magnetic field at right angles to rotor magnetization for a first half of a time interval between successive stops to facilitate acceleration, reversing the magnetic field for the second half of the time interval between successive stops to facilitate deceleration, applying the magnetic field along the new rotor position to retain positioning for the time allotted for imaging at that position, and repeating the previous steps, as needed during an imaging procedure. It will be understood that torque may be applied to the magnetic field at other angles relative to the rotor magnetization vector other than at right angles to the rotor magnetization vector or in the same direction as the rotor magnetization vector.

While not wishing to be bound by any particular theory, in at least some embodiments, the magnetic torque τ exerted on the magnet 424 is given by:

τ=m×H=mH sin(θ)k; (A)

where τ=the torque vector in N-m; m=the magnetic moment vector in Tesla-m³; H=the magnetic field vector of the windings 502 and 504 in amp/m; θ=the angle between the magnetic moment and magnetic field; and k=the unit vector directed along the motor axis.

The magnetic moment vector m is given by:

m=MV=(π/4)(D₂²−D₁²)LM; (B)

where M=the magnetization vector of the magnet 424 in Tesla; V=the volume of the magnet 424 in m³; D₂=the outside diameter of the magnet 424 in m; D₁=the inside diameter of the magnet 424 in m; and L=the length of the longitudinal axis 428 of the magnet 424 in m.

The magnetic field H of the three-phase strip line stator winding is given by:

H=3I/(2π/D_{w}); (C)

where H=the magnetic field in Amps/m; I=the current in the windings 502 and 504 in Amps; and D_{w}=the diameter of the windings 502 and 504 in m.

Combining formula (B) and (C), the torque on the magnet 424 may be given by:

τ=(3/8D_{w})MI(D₂²−D₁²)L sin(θ); (D)

Acceleration of the magnet 424 and the resulting angular displacement of the applied magnetic field may be computed by setting the torque to be equal to the moment of inertia of the magnet 424 times its angular acceleration. At least one previous experiment has shown that friction on the magnet 424 is negligible during the acceleration phase because the magnet 424 starts and stops with nearly equal acceleration and deceleration times.

The moment of inertia of the magnet 424 about its longitudinal axis 428 is given by:

I=(1/8)N(D₂²+D₁²)=(π/32)ρL(D₂⁴−D₁⁴); (E)

where I=the moment of inertia of the magnet 424 in kg-m²; N=the mass of the magnet 424 in kg; and ρ=the density of the magnet 424 in kg/m³.

The equation of motion of the magnet 424 (neglecting friction) is given by:

Id²φ/dt²=τ; (F)

where t=time in sec; and φ=the angle of the magnet 424 in radians.

Using the formula (D), the torque is maximum when the magnetic field is applied at an angle that is 90 degrees (at 90 degrees, sin(θ)=1) from the magnetization of the magnet 424.

This remains approximately true over the size (1.4 degrees) of the angular displacements of the magnet 424 considered herein.

Substituting formulas (D) and (E) into formula (F) and integrating, the angle of the magnet 424 is given by:

φ=½αt²; (G)

where α=the angular acceleration in radians/sec²; and where:

α=12MI/[πρD_{w}{D₂²+D₁²}]. (H)

Accordingly, formula (H) shows that the acceleration of the magnet 424 is linear in applied current and inversely proportional to the cube of the diameter of the motor 412. Additionally, formula (H) shows that the acceleration of the magnet 424 is independent of the length of the longitudinal axis 428 of the magnet 424.

When the motor 412 is starting and stopping at regular intervals (e.g., during an imaging procedure), acceleration is applied for a period of time to reach the angle given by formula (G), and then deceleration of the same magnitude is applied for the same amount of time to stop the magnet 424. The total angular displacement is equal to two times the displacement that occurs during acceleration of the magnet 424. For example, when the motor 412 is configured and arranged to stop 256 times at equal intervals during one rotation, each stop has an angular displacement of 1.4 degrees (360 degrees divided by 256 degrees). For example, at 30 Hz the motor 412 has approximately 100 microseconds to travel between successive stops of 30 microseconds each. Thus, during the acceleration phase, the magnetic field needs to be displaced 0.7 degrees over 50 microseconds. The deceleration phase would similarly displace the magnetic field 0.7 degrees over 50 microseconds.

In one experiment, the motor rotation algorithm was applied to a one-millimeter diameter magnetic motor with a three-phase winding. The motor rotation algorithm included repeated application of a magnetic field at right angles to rotor magnetization for a first half of a time interval between successive stops, followed by reversal of the magnetic field for the second half of the time interval between successive stops to facilitate deceleration, followed by a retention of the magnet at a current position. The motor rotation algorithm was implemented in machine language and applied to fast digital-to-analog convertors to control a current with an amplitude of 7 Amps that was applied to the three-phase winding.

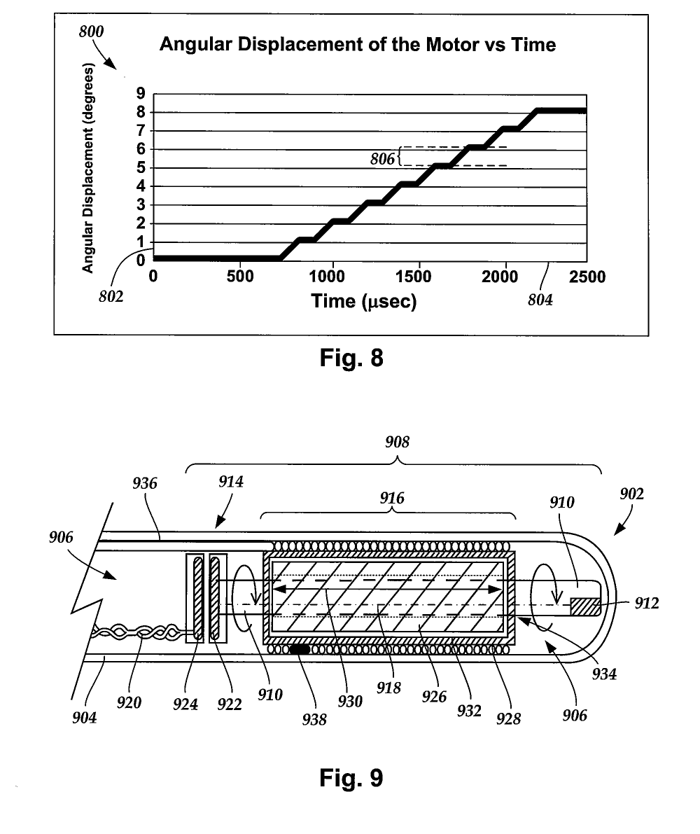

FIG. 8 is a graph 800 of the angular displacement 802 of a one-millimeter diameter motor over time 804. The motor was advanced along eight one-degree increments 806, with a 65 microsecond stop time between each advancement. The prolonged stop time was used to more clearly show the incremental movement of the motor. An acceleration vector was applied at right angles to the rotor magnetization vector of the magnet for 55 microseconds, then reversed for 55 microseconds.

As shown in the graph 800 of FIG. 8, approximately 0.5 degrees of rotor angular displacement occurred in a 55 microsecond acceleration period. This result can be verified by inputting appropriate values for a one-millimeter diameter motor into formula (G). For example, inputting the values: M≈1 T; I=7 Amps; ρ=5,000 kg/m³; D_{w}=0.001 m; D₁=0.0003 m; D₂=0.0008 m; and t=55×10⁶ sec into formula (G), and then converting φ from radians to degrees results in φ≈0.6 degrees, which is in agreement with the measured value for φ of approximately 0.5 degrees, recorded in the graph 800 of FIG. 8.

When a medical device, such as an IVUS system, is inserted into a patient, it is typically important to prevent undue heating of the inserted device to prevent undesired patient injury. In at least some embodiments, the applied current may be adjusted to prevent excessive heating by the motor 412. In at least some embodiments, the diameter of the motor may be reduced, as expressed in Equation (H), to reduce the current required to achieve a given angular acceleration, thus reducing the heat generated by the motor to safe levels.

The amount of magnetic torque that may be generated by the motor 416 may be limited by the amount of current that may be passed through the windings 502 and 504 without generating excessive heat in the catheter (402 in FIG. 4). Heat is generated in the windings 502 and 504 by Joule heating at a rate given by:

P=I²R;

where P=the power dissipated as heat in watts; R=the resistance of the windings 502 and 504; and I=the amplitude of the current in Amps.

The value for P is divided by two because sinusoidal current is employed. However the value for P is also multiplied by two because there are two windings 502 and 504. In at least some instances, it has been estimated that up to 300 mW of heat is readily dissipated in blood or tissue without perceptibly increasing the temperature of the motor (416 in FIG. 4). In at least one experiment, it has been estimated that heat dissipation increases to several watts when blood is flowing.

In at least some embodiments, the imaging core is configured and arranged such that the rotatable stepper motor causes a transducer to rotate. FIG. 9 is a schematic longitudinal cross-sectional view of one embodiment of a distal end of a catheter 902. The catheter 902 includes a sheath 904 and a lumen 906. A rotatable imaging core 908 is disposed in the lumen 906 at the distal end of the catheter 902. The imaging core 908 includes a rotatable driveshaft 910 with one or more transducers 912 coupled to a distal end of the driveshaft 910 and a transformer 914 coupled to a proximal end of the driveshaft 910. The imaging core 908 also includes a motor 916 coupled to the driveshaft 910. One or more imaging core conductors 918 electrically couple the one or more transducers 912 to the transformer 914. In at least some embodiments, the one or more imaging core conductors 918 extend within the driveshaft 910. One or more transducer conductors 920 electrically couple the transformer 914 to the control module (104 in FIG. 1). In at least some embodiments, the one or more of the transducer conductors 920 may extend along at least a portion of the longitudinal length of the catheter 902 as shielded electrical cables, such as a coaxial cable, or a twisted pair cable, or the like.

The transformer 914 is disposed on the imaging core 908. In at least some embodiments, the transformer 914 includes a rotating component 922 coupled to the driveshaft 910 and a stationary component 924 disposed spaced apart from the rotating component 914. In some embodiments, the stationary part 924 is proximal to, and immediately adjacent to, the rotating component 922. The rotating component 922 is electrically coupled to the one or more transducers 912 via the one or more imaging core conductors 918 disposed in the imaging core 908. The stationary component 916 is electrically coupled to the control module (104 in FIG. 1) via one or more conductors 920 disposed in the lumen 906. Current is inductively passed between the rotating component 922 and the stationary component 924 (e.g., a rotor and a stator, or a rotating pancake coil and a stationary pancake coil, or the like).

In at least some embodiments, the transformer 914 is positioned at a proximal end of the imaging core 908. In at least some embodiments, the components 922 and 924 of the transformer 914 are disposed in a ferrite form. In at least some embodiments, the components 922 and 924 are smaller in size than components conventionally positioned at the proximal end of the catheter.

The motor 916 includes a rotor 926 and a stator 928. In at least some embodiments, the rotor 926 is a permanent magnet with a longitudinal axis, indicated by a two-headed arrow 930, which is coaxial with the longitudinal axis of the imaging core 908 and the driveshaft 910. The motor 916 may be formed from similar materials, and with similar magnetization, as magnet 424, discussed above. In at least some embodiments, the magnet 926 is cylindrical. In at least some embodiments, the magnet 926 is disposed in a housing 932.

In at least some embodiments, the magnet 926 is coupled to the driveshaft 910 and is configured and arranged to rotate the driveshaft 910 during operation. In at least some embodiments, the magnet 926 defines an aperture 934 along the longitudinal axis 930 of the magnet 926. In at least some embodiments, the driveshaft 910 and the one or more imaging core conductors 918 extend through the aperture 934. In at least some other embodiments, the drive shaft 910 is discontinuous and, for example, couples to the magnet 926 at opposing ends of the magnet 926. In which case, the one or more imaging core conductors 918 still extend through the aperture 934. In at least some embodiments, the magnet 926 is coupled to the driveshaft 910 by an adhesive. Alternatively, in some embodiments the driveshaft 910 and the magnet 926 can be machined from a single block to magnetic material with the aperture 934 drilled down a length of the driveshaft 910 for receiving the imaging core conductors 918.

In at least some embodiments, the stator 928 includes two perpendicularly-oriented magnetic field windings (502 and 504 in FIG. 5) which provide a rotating magnetic field to produce torque causing rotation of the magnet 926. The stator 928 is provided with power from the control module (104 in FIG. 1) via one or more motor conductors 936. In at least some embodiments, a sensing device 938 is disposed on the imaging core 908. In at least some embodiments, the sensing device 938 is coupled on the housing 932.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A catheter assembly for an intravascular ultrasound system, the catheter assembly comprising:
a catheter having a longitudinal length, a distal end, and a proximal end, the catheter comprising a lumen extending along at least a portion of the catheter;
an imaging core with a longitudinal length that is substantially less than the longitudinal length of the catheter, the imaging core configured and arranged for insertion into the lumen of the catheter and disposition at the distal end of the catheter, the imaging core comprising
a rotatable driveshaft having a distal end and a proximal end,
a mirror disposed at the distal end of the driveshaft such that rotation of the driveshaft causes a corresponding rotation of the mirror,
a stepper motor coupled to the proximal end of the driveshaft and configured and arranged to provide step-wise rotation of the driveshaft, the stepper motor comprising a rotatable magnet and at least two magnetic field windings disposed around at least a portion of the magnet, and
at least one fixed transducer positioned between the stepper motor and the mirror, the at least one transducer having an aperture defined along a longitudinal axis of the at least one transducer, the aperture configured and arranged to allow passage of the driveshaft through the at least one transducer to the rotatable mirror, the at least one transducer configured and arranged for transforming applied electrical signals to acoustic signals, transmitting the acoustic signals, receiving corresponding echo signals, and transforming the received echo signals to electrical signals;
at least one transducer conductor electrically coupled to the at least one transducer and in electrical communication with the proximal end of the catheter; and
at least one motor conductor electrically coupled to the magnetic field windings and in electrical communication with the proximal end of the catheter.

2. The catheter assembly of claim 1, wherein the stepper motor is configured and arranged to rotate the magnet such that the magnet completes at least 20 360-degree cycles per second.

3. The catheter assembly of claim 1, wherein the stepper motor is configured and arranged to permit stepwise rotation of the driveshaft with steps of 3 degrees or less.

4. The catheter assembly of claim 1, wherein the stepper motor is configured and arranged to permit stepwise rotation of the driveshaft with steps of 2 degrees or less.

5. The catheter assembly of claim 1, wherein the mirror is tilted at an angle such that when an acoustic beam is emitted from the at least one transducer to the mirror, the acoustic beam is redirected in a direction that is not parallel the longitudinal axis of the magnet.

6. The catheter assembly of claim 1, wherein the magnetic field windings are disposed on a rigid slotted material.

7. The catheter assembly of claim 1, wherein the imaging core further comprises a sensing device, the sensing device configured and arranged for sensing an angular position of the magnet.

8. The catheter assembly of claim 1, wherein the motor has a transverse outer diameter that is no more than 0.5 millimeters.

9. The catheter assembly of claim 1, wherein the mirror is disposed within sonolucent material having an impedance within 10 percent of an impedance of patient tissue or fluids in proximity to the distal end of the catheter, and wherein the sonolucent material is positioned to have an even weight distribution around the driveshaft.

10. An intravascular ultrasound imaging system comprising:
the catheter assembly of claim 1; and
a control module coupled to the imaging core, the control module comprising
a pulse generator configured and arranged for providing electric signals to the at least one transducer, the pulse generator electrically coupled to the at least one transducer via the at least one transducer conductor, and
a processor configured and arranged for processing received electrical signals from the at least one transducer to form at least one image, the processor electrically coupled to the at least one transducer via the at least one transducer conductor.

11. A catheter assembly for an intravascular ultrasound system, the catheter assembly comprising:
a catheter having a longitudinal length, a distal end, and a proximal end, the catheter comprising a lumen extending along at least a portion of the catheter;
an imaging core with a longitudinal length that is substantially less than the longitudinal length of the catheter, the imaging core configured and arranged for insertion into the lumen of the catheter and disposition at the distal end of the catheter, the imaging core comprising
a rotatable driveshaft having a distal end and a proximal end,
at least one transducer disposed at the distal end of the driveshaft such that rotation of the driveshaft causes a subsequent rotation of the at least one transducer, the at least one transducer configured and arranged for transforming applied electrical signals to acoustic signals, transmitting the acoustic signals, receiving corresponding echo signals, and transforming the received echo signals to electrical signals,
a transformer disposed at the proximal end of the driveshaft,
at least one imaging core conductor coupling the at least one transducer to the transformer, and
a stepper motor coupled to the driveshaft between the one or more transducers and the transformer, the stepper motor configured and arranged to produce step-wise rotation of the driveshaft, the stepper motor comprising a rotatable magnet and at least two magnetic field windings disposed around at least a portion of the magnet, the magnet having a longitudinal axis and an aperture defined along at least a portion of the longitudinal axis of the magnet;
at least one transducer conductor electrically coupled to the transformer and extending to the proximal end of the catheter; and
at least one motor conductor electrically coupled to the magnetic field windings and extending to the proximal end of the catheter.

12. The catheter assembly of claim 11, wherein the stepper motor is configured and arranged to produce step-wise rotation of the driveshaft with steps of 3 degrees or less

13. The catheter assembly of claim 11, wherein at least one of the at least one imaging core conductor or the driveshaft extends through the aperture of the magnet.

14. An intravascular ultrasound imaging system comprising:
the catheter assembly of claim 11; and
a control module coupled to the imaging core, the control module comprising
a pulse generator configured and arranged for providing electric signals to the at least one transducer, the pulse generator electrically coupled to the at least one transducer via the one or more conductors and the transformer, and
a processor configured and arranged for processing received electrical signals from the at least one transducer to form at least one image, the processor electrically coupled to the at least one transducer via the one or more conductors.

15. A method for imaging a patient using an intravascular ultrasound imaging system, the method comprising:
a) inserting a catheter into patient vasculature, the catheter having a longitudinal axis and comprising an imaging core disposed in a distal portion of a lumen defined in the catheter, the imaging core electrically coupled to a control module by at least one conductor, the imaging core having a longitudinal axis and comprising at least one transducer, a driveshaft, and a magnet that rotates the driveshaft by application of a current from the control module to at least two magnetic field windings wrapped around at least a portion of the magnet, wherein the transducer emits acoustic signals directed at patient tissue, and wherein the rotation of the magnet causes rotation of the driveshaft;
b) positioning the imaging core in a region to be imaged;
c) applying an electrical signal to the at least two magnetic field windings to generate rotational acceleration of the magnet for a period of time of acceleration sufficient for the magnet to rotate by a selected amount;
d) applying an electrical signal to the at least two magnetic field windings to generate rotational deceleration of the magnet for a period of time of deceleration that is equal to the period of time of acceleration;
e) applying an electrical signal to the at least two magnetic field windings to generate the electrical signal causing the magnet to maintain a fixed position for a period of time;
f) transmitting at least one acoustic signal from the at least one transducer to patient tissue during the period of time when the magnet is maintained in the fixed position;
g) receiving at least one echo signal during the period of time when the magnet is maintained in the fixed position; and
h) repeating steps c) through g) until the magnet has rotated at least one 360-degree cycle around the longitudinal axis of the imaging core.

16. The method of claim 15, wherein repeating steps c) through g) comprises moving the imaging core along the longitudinal axis of the catheter after performing the steps c) through g).

17. The method of claim 15, wherein inserting the catheter into patient vasculature comprises inserting the catheter into patient vasculature, wherein the at least one transducer is fixed, wherein the imaging core further comprises a tilted mirror coupled to the rotatable driveshaft, and wherein the tilted mirror is configured and arranged to reflect the at least one acoustic signal transmitted from the at least one fixed transducer to patient tissue and also to redirect the at least one echo signal received from patient tissue to the at least one transducer.

18. The method of claim 15, wherein inserting the catheter into patient vasculature comprises inserting the catheter into patient vasculature, wherein the at least one transducer is coupled to the rotatable driveshaft.

19. The method of claim 15, wherein transmitting at least one electrical signal from the control module to the at least two magnetic field windings comprises transmitting at least one electrical signal that causes rotational acceleration of the magnet for a period of time sufficient for the magnet to rotate 1.5 degrees or less.

20. The method of claim 19, wherein applying an electrical signal to the at least two magnetic field windings to generate the electrical signal causing the magnet to maintain a fixed position for a period of time comprises applying an electrical signal to the at least two magnetic field windings to generate the electrical signal causing the magnet to maintain a fixed position for a period of time of no more than 50 microseconds.
